(19) United States
(12) Patent Application Publication
(10) Pub. No.: US 2010/0080790 A1
(43) Pub. Date: Apr. 1, 2010

(54) STERILIZATION USING HIGH-PRESSURE CARBON DIOXIDE

(75) Inventors: Michael A. Matthews, Blythewood, SC (US); Jian Zhang, West Columbia, SC (US)

Correspondence Address: Ballard Spahr LLP, SUITE 1000, 999 PEACHTREE STREET, ATLANTA, GA, 30309-3915, US

(73) Assignee: University of South Carolina

(21) Appl. No.: 11/995,654

(22) PCT Filed: Jul. 7, 2006
(86) PCT No.: PCT/US06/26412

§ 371 Date: Apr. 26, 2009

Related U.S. Application Data
(60) Provisional application No. 60/699,007, filed on Jul. 13, 2005.

(51) Int. Cl. A61L 2/18; A01N 59/04; A01N 63/00; A01P 1/00; A61L 2/00

(52) U.S. Cl. 424/94.61; 424/700; 422/33

(57) ABSTRACT

Disclosed are compositions and methods for the biocompatible sterilization of materials, in particular, of medical devices and implants. Sterilization is achieved by deactivation of microorganisms through treatment of the material with a mixture of at least one microbiocidal additive and a high-pressure or supercritical fluid, for example, high-pressure carbon dioxide or supercritical carbon dioxide. This abstract is intended as a scanning tool for purposes of searching in the particular art and is not intended to be limiting of the present invention.

### CROSS-REFERENCE TO RELATED APPLICATIONS

This application claims the benefit of U.S. Application No. 60/699,007, filed Jul. 13, 2005, which is hereby incorporated herein by reference in its entirety.

### ACKNOWLEDGEMENT

This invention was made with government support under Grant R01 EB 055201 awarded by the National Institutes of Health. The government has certain rights in the invention.

### BACKGROUND

Sterilization of medical devices that are used in intimate contact with the human body is crucial to aid in prevention of patient infection. Sterilization of medical devices and implants is a serious issue for surgical wards and hospitals. For example, in the United States, over 600,000 arthroplasties are performed each year, of which 0.6-2.3% result in infection. These infections can cause substantial physical injury or even death to the patient. Some other widely used medical devices, such as endoscopes, can also cause infection if not properly sterilized. Disinfection of heat-sensitive biomaterials, especially polymers, presents a challenge to the currently used techniques of microorganism destruction. There is increasing interest in the use of biopolymers, autograft tissue, and allograft tissue in, for example, regenerative medicine, and these materials generally require sterilization prior to implantation.

Current methods of sterilization typically employ high temperatures, toxic chemicals, strong radiation, or strongly oxidizing chemical additives that are detrimental to such materials and that may corrode or damage the materials of construction of biomedical devices and sterilization equipment.

In medical practice, standard sterilization methods include steam sterilization, gamma-irradiation, ethylene oxide, and hydrogen peroxide sterilization. Most of these techniques can have serious drawbacks. For example, steam autoclaving damages heat-sensitive materials and deposits an oxide layer onto metallic surfaces. Gamma-irradiation reduces shear and tensile strength, elastic modulus, and transparency of medical polymers by breaking polymer chains and the resultant reactions of the free radicals produced. Ethylene oxide not only changes the material properties of polymers but also requires special safety considerations because of its flammability and toxicity. Hydrogen peroxide is recognized as a sterilant only when used in relatively high concentration in aqueous solution, and with relatively long contact times. The aqueous solution itself is a strong irritant. Because of the various drawbacks associated with current sterilization techniques, the next generation of polymeric medical devices and heat sensitive biomaterials call for the use of new sterilization methods.

Treatment with liquid or supercritical carbon dioxide (CO₂) in the dense state can be an effective way to destroy certain vegetative bacteria; however, several species of spores, such as Bacillus subtilis and Geobacillus stearothermophilus, have proven to be highly resistant to sterilization methods with high-pressure CO₂ alone. Due to the high resistance of spores to treatment with pure CO₂, some combination of elevated pressure, high temperature, or extended treatment time are typically required to achieve a significant reduction in the number of surviving, active spores. However, excessively high pressure and/or temperature can damage heat-sensitive materials and devices, and increase investment and operating cost. Therefore, it can be desirable to operate the sterilization process at a relatively low temperature and pressure, and for a relatively short period of time.

Therefore, there remains a need for methods and compositions that overcome these deficiencies and that effectively sterilize bacteria and bacterial spores.

### SUMMARY

Disclosed are methods and compositions related to sterilization of microorganisms including but not limited to bacterial spores with compositions comprising the combination of pressurized carbon dioxide and microbiocidal additives.

Also disclosed are methods for sterilizing a material having microorganisms to be inactivated comprising the steps of contacting the material with a mixture comprising a non-oxidative microbiocidal additive and high-pressure carbon dioxide or supercritical carbon dioxide, and maintaining the contact for a period of time effective to achieve a degree of inactivation of the microorganisms exceeding 2 log orders.

Also disclosed are methods for sterilizing a material having microorganisms to be inactivated comprising the steps of contacting the material with a mixture comprising hydrogen peroxide and supercritical carbon dioxide, and maintaining the contact for a period of time effective to achieve a degree of inactivation of the microorganisms exceeding 2 log orders.

Also disclosed are compositions for sterilizing a material, comprising a non-oxidative microbiocidal additive and high-pressure carbon dioxide or supercritical carbon dioxide.

Also disclosed are methods for sterilizing a material having microorganisms to be inactivated comprising the steps of contacting the material with a mixture comprising a non-oxidative microbiocidal additive and a high-pressure or supercritical fluid, and maintaining the contact for a period of time effective to achieve a degree of inactivation of the microorganisms exceeding 2 log orders.

Also disclosed are compositions for sterilizing a material comprising a non-oxidative microbiocidal additive and a high-pressure or supercritical fluid.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the effect of treatment time using CO₂ on the deactivation of the water-wetted spores.

FIG. 2 shows a schematic of an exemplary high-pressure or supercritical fluid apparatus.

### DETAILED DESCRIPTION

Before the present compounds, compositions, articles, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “an additive” includes mixtures of two or more such additives, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data are provided in a number of different formats and that these data represent endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

“High-pressure carbon dioxide,” “high-pressure CO₂,” “dense-phase carbon dioxide,” and “dense-phase CO₂” refer to pressurized, liquid carbon dioxide near but below the critical temperature and near but below the critical pressure. For example, in one aspect, the pressure can be from about 400 pounds per square inch (about 27.6 bar) to about 1,070 pounds per square inch (about 73.7 bar). In another aspect, the pressure can be from about 500 pounds per square inch (about 34.5 bar) to about 850 pounds per square inch (about 58.6 bar). In a further aspect, the pressure can be from about 600 pounds per square inch (about 41.36 bar) to about 750 pounds per square inch (about 51.7 bar).

“Supercritical CO₂” refers to pressurized, fluid carbon dioxide at or above the critical temperature (about 31.1° C.) and at or above the critical pressure (about 73.8 bar).

“High-pressure fluid” and “dense-phase fluid” refer to any pressurized liquid near but below its critical temperature and near but below its critical pressure. In one aspect, the pressure can be from 35% to 99% of the critical pressure of the fluid, for example, from 40% to 85% of the critical pressure, for example, from 60% to 75% of the critical pressure.

“Supercritical fluid” refers to a pressurized fluid at or above its critical temperature and at or above its critical pressure.

“Microbiocidal” refers to having the property of inactivating pathogens or any microorganisms.

“Microbiocidal additive” or “microbiocidal agent” as used herein, refers to having the property of inactivating pathogens when used as an additive in a high-pressure or supercritical fluid, in particular carbon dioxide.

“Microorganisms,” as used herein, refers to and is understood to include active biological contaminants or pathogens, including bacteria (including inter- and intracellular bacteria, such as mycoplasmas, ureaplasmas, nanobacteria, chlamydia, rickettsias), yeasts, molds, fungi, spores, or similar agents and/or single or multicellular parasites, and combinations thereof.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. STERILIZATION METHODS GENERALLY

The degree of deactivation of microorganisms by a sterilization method can be characterized by log reduction. Log reduction can be calculated with the following equation:

$$Log Reduction = log ( \frac{number of untreated microorganisms}{number of surviving microorganisms after treatment} )$$

Typically, deactivation methods employing high pressure CO₂ or supercritical CO₂ can deactivate B. pumilus spores by up to three-log. However, at least six-log reduction is required by the Federal Drug Administration (FDA) to claim sterilization. Therefore, carbon dioxide and water alone do not achieve adequate deactivation (Table 1).

TABLE 1
LOG REDUCTION OF B. PUMILUS SPORES ON WETTED
SPORE STRIPS WITH CO₂.

| DURATION | PRESSURE | TEMPERATURE | |
|---|---|---|---|
| [HR] | [BAR] | [° C.] | LOG REDUCTION |
| 4 | 276 | 50 | 0.58 ± 0.04 |
| | 276 | 60 | 3.06 ± 0.17 |
| | 276 | 80 | 3.02 ± 0.07 |
| | 103 | 60 | 1.91 ± 0.23 |

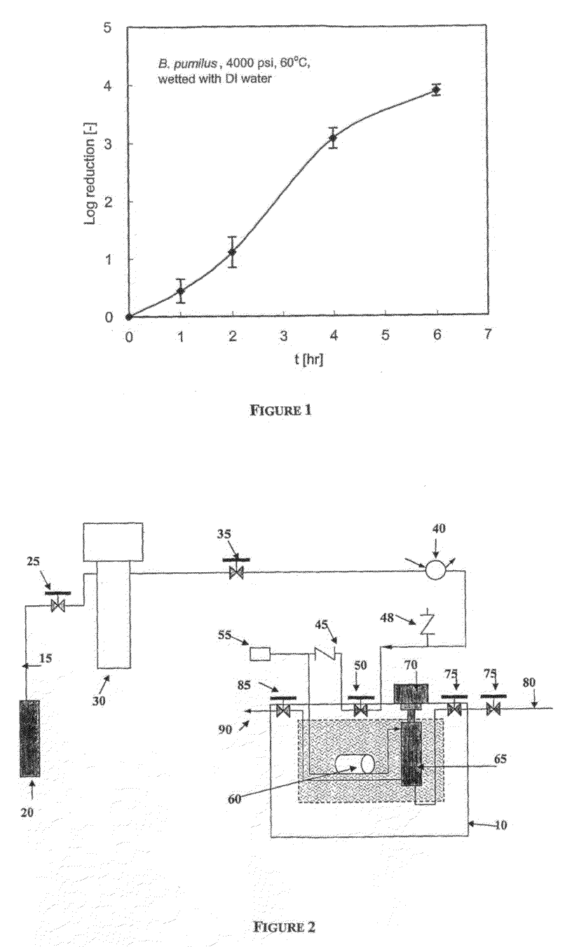

The effect of exposure time to CO₂ on the deactivation of the water-wetted spores is shown in FIG. 1. The nonlinear curve in FIG. 1 can be divided into three stages. In the first stage (0-2 hours) the log reduction increases slowly with the treatment time, while in the second stage (2-4 hours) the deactivation proceeds more rapidly. In the last stage (>4 hours), the log reduction appears to approach a limiting value. After six hours of treatment, only a 3.90-log reduction had been obtained in the water-wetted spores. Such a long process time is generally not ideal for a commercial sterilizer.

### C. IMPROVED STERILIZATION METHODS

The present invention relates to the sterilization and disinfection arts. It finds particular application in conjunction with high-pressure fluids and supercritical fluids associated with antimicrobial agents, such as sterilants or disinfectants, for combined cleaning and sterilization or disinfection of medical instruments, equipment, and supplies, and will be described with particular reference thereto. It should be appreciated, however, that the invention is also applicable to the sterilization or disinfection of other items, including food processing equipment and packaging and hospital supplies, such as bed linen and protective clothing, and the like.

The disclosed compositions and methods can be used, generally, for any surface having microorganisms. For example, the disclosed compositions and methods can be used with medical equipment including, without limitation, surgical instruments, devices for implants, cannulas, endoscopes, syringes, bandages, medical packaging, and vials. The disclosed compositions and methods can also be used with tissue. The disclosed compositions and methods can also be used with food processing equipment including, without limitation, eating utensils, cookware, and food and beverage containers. The disclosed compositions and methods can be also used with personal items including, without limitation, clothing, bedding, hospital and institutional bedding and draperies, hospital and institutional towels, contact lenses, grooming supplies, and jewelry.

The disclosed compositions and methods can be used, generally, with substrates of any material. Materials suitable for use with the disclosed compositions and methods include, without limitation, metals such as aluminum, iron, stainless steel, titanium, gold, silver, platinum, and mixtures thereof; plastics such as polyesters, nylons, polyolefins, and mixtures thereof; glass; stone; ceramics; and mixtures thereof.

The disclosed compositions and methods can achieve deactivation of microorganisms, including bacterial spores, resulting in a log reduction of, for example, greater than or equal to: 1-log, 2-log, 3-log, 4-log, 5-log, 6-log, or 7-log.

1. Sterilizing Fluids

a. Carbon Dioxide

Carbon dioxide can be used in the disclosed method and compositions as a high-pressure, dense or as a supercritical fluid. A supercritical fluid is a pure fluid or mixture of fluids which is at a temperature and pressure at or above its critical temperature and pressure.

High-pressure sterilization involves temperatures near but below the critical temperature and pressures near but below the critical pressure. Generally, such high-pressure fluid can be in the liquid state, typically from 35% to 99% of the critical pressure of the fluids. For carbon dioxide, sufficiently high pressures can be below about 73.8 bar, the critical pressure of carbon dioxide.

Supercritical sterilization employs temperatures at or above the critical temperature and pressures at or above the critical pressure. The critical temperature and pressure vary with the fluid selected. The critical temperature of a fluid is the temperature above which the fluid can no longer be liquefied, irrespective of the pressure applied. The critical pressure is the pressure at which a substance may exist as a gas in equilibrium with a liquid at the critical temperature. Thus, the properties of a dense fluid change appreciably at or above the critical pressure.

Carbon dioxide is a particularly advantageous fluid because it is a non-polar solvent. This allows co-solvents to be added having a high degree of selectivity. For carbon dioxide, the critical pressure is 73.8 bar and the critical temperature is 31.1° C.

Using CO₂ as a fluid to effect sterilization has several potential benefits. First, CO₂ is not flammable and is non-toxic; the chief hazard in its use is asphyxiation. Unlike ethylene oxide, CO₂ requires no special handling or ventilation, and leaves no toxic residues. Second, CO₂ is inert in most situations so it does not react with polymers, which alleviates the aging problem caused by γ-irradiation. Also, CO₂ has a low critical temperature (31.1° C.). This is only slightly above room temperature, so thermal degradation is not a problem when a process is operated around the critical temperature. In a supercritical state, CO₂ has low viscosity (about 3 to 7×10⁻⁵N·s·m⁻²) and extremely low or zero surface tension, so it can quickly penetrate complex structure and porous materials.

b. Other Fluids

Other fluids, at high-pressure or supercritical pressure, can be used to deactivate microorganisms, including bacterial spores. These fluids can include, but are not limited to, nitrous oxide, ethylene, tetrafluoroethane (TFE), and mixtures thereof.

Because N₂ is far from its critical point (T_{c}=−147° C., P_{c}=34 bar) at normal ambient conditions, it does not have the unique properties of gas-like diffusivity and liquid-like density, which make CO₂ more effective under the same conditions.

TFE has a similar critical point (T_{c}=328 K, P_{c}=41 bar) to CO₂ (T_{c}=304.13 K, P_{c}=73.8 bar), but has different chemical properties (dipole moment, D_{CO2}=0 D, D_{TFE}=1.80±0.22 D; solubility parameter, δ_{CO2}=7.0, δ_{TFE}=13.6).

Supercritical N₂O is more effective than N₂ in recovering nucleic acids from vegetative bacteria and yeast. This can be due to the high density and low polarity of supercritical N₂O, which might solubilize lipids and hydrophobic compounds in the cell wall and the cytoplasmic membrane. N₂O's critical parameters are very close to those of CO₂. N₂O only has a small dipole moment, while CO₂ has a zero dipole moment; both have a comparatively high solubility in water.

It is understood that the disclosed methods can optionally be performed with fluids other than high-pressure carbon dioxide, for example, with nitrous oxide, ethylene, tetrafluoroethane, and mixtures thereof. It is also understood that the disclosed methods can be performed with high-pressure or supercritical carbon dioxide in combination with these other fluids.

2. Non-Oxidative Methods

Certain natural biomolecules (e.g., enzymes) and synthetic macrocyclic hydraphiles can attack the membrane layers of E. coli, G. stearothermophilus, or S. aureus. Such compounds will attack biological membranes and barriers selectively, and are not destructive to metals, polymers, and ordinary materials of construction. The mechanism of sterilization with these molecules does not involve oxidation, heat, or radiation. Collectively, such additives can be referred to as non-oxidative microbiocidal additives.

It is understood that the disclosed composition and methods can optionally include non-oxidizing agents as a microbiocidal additive. Suitable non-oxidative microbiocidal agents include, for example, spore germinants, peptidoglycan hydrolyzers, ion channel forming compounds, and mixtures thereof. It is also understood that other non-oxidizing agents known to those of skill in the art can be used in the disclosed methods and compositions.

In one aspect, the non-oxidizing microbiocidal agent can be added to the disclosed compositions and used in the disclosed methods in a concentration of greater than about 1000 ppm of the total composition. In another aspect, the concentration of non-oxidizing agent can be less than about 1000 ppm of the total composition, for example, less than about 800 ppm, less than about 600 ppm, less than about 500 ppm, less than about 400 ppm, less than about 300 ppm, less than about 200 ppm, less than about 100 ppm, less than about 50 ppm, less than about 25 ppm, less than about 10 ppm, or less than 5 ppm.

In a further aspect, the concentration can be from about 5 ppm to about 1000 ppm, from about 10 ppm to about 800 ppm, from about 25 ppm to about 500 ppm, from about 50 ppm to about 400 ppm, from about 50 ppm to about 300 ppm, from about 50 ppm to about 200 ppm, from about 75 ppm to about 125 ppm, about 50 ppm, about 100 ppm, about 150 ppm, or about 200 ppm.

In yet another aspect, the composition can be substantially free of non-oxidizing microbiocidal agent.

a. Spore Germinants

Spore germinants can facilitate deactivation of microorganisms, including bacterial spores. By inducing germination of sterilization-resistant spores, germinants enhance high-pressure and supercritical carbon dioxide sterilization of bacterial spores.

Examples of spore germinants are L-alanine and a class of germinants composed of an asparagine, dipicolinic acid, glucose, fructose, or a potassium ion, as disclosed by Wuytack, E. Y., Boven, S., and Michiels, C. W., “Comparative study of pressure-induced germination of Bacillus subtilis spores at low and high pressures,” Appl. Environ. Microbiol., 64 (9) (1998) 3220, hereby specifically incorporated herein by reference.

By providing spores with a germinant to initiate the germination process, the resistance of the spore becomes weaker. The pathway of dormant spores to activated spores to deactivated spores is an effective way to lessen the high resistance of dormant spores. Dipicolinic acid (DPA) can trigger the L-alanine-induced germination on Ca²⁺ and/or DPA deprived B. cereus spores by facilitating the transfer of L-alanine to germination sites.

Spore germinants can optionally be used in the disclosed methods and compositions. It is also understood that the present invention can be performed using other spore germinants known to those of skill in the art.

b. Peptidoglycan Hydrolyzers

Lysozyme, a small protein existing in tears, saliva, and blood serum. Lysozyme is an enzyme which can hydrolyze peptidoglycan. The thick peptidoglycan cortex is a source of spores' high resistance. Although lysosyme is small relative to other members of the protein family, its molecular weight is still over 14,400, so its solubility in CO₂ is generally very low. For example, CO₂ can be used as an antisolvent to produce lysozyme particles. A reverse micelle of lysozyme/surfactant can be successfully suspended in the CO₂ phase, but introducing a surfactant into the system can require later removal of that surfactant following sterilization.

An alternative can be to use a molecule that is smaller than lysozyme but that retains its microbiocidal properties. A lysozyme-derived peptide, HEL96-116, which is a peptide of 15 amino-acids, can be an effective deactivator in high hydrostatic pressure treatment on microorganisms.

Peptidoglycan hydrolyzers can optionally be used in the disclosed methods and compositions. Suitable peptidoglycan hydrolyzers include, without limitation, lysozyme, lysostaphin, autolysin, HEL96-116 peptide, or a mixture thereof. It is understood that the present invention can be performed using other peptidoglycan hydrolyzers known to those of skill in the art.

c. Ion Channel Forming Compounds

Macrocycle-based compounds called hydraphiles can form channels and conduct cations across bilayer membranes. These compounds can be crown ether-based synthetic cation conducting channels and can also be active deactivating agents against bacterium. Hydraphiles can be toxic to microorganisms as a result of channel formation in the membrane.

Hydraphiles can insert into the cell bilayer and disrupt the osmotic balance, leading to cell death. Microbiocidal activity can depend upon the presence of a functional central relay and proper channel length. For example, a C12 hydrophile, which spans the bilayer, can be 13 times more active against Escherichia coli than compounds forming shorter channels.

An example of a hydraphile that can be used in conjunction with the present invention has the following structure:

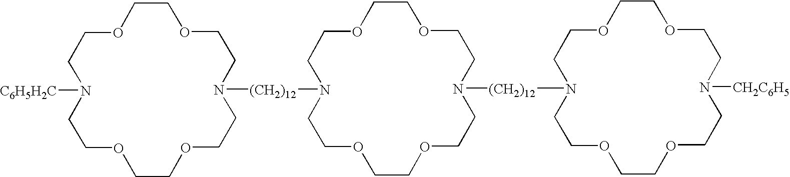

It is understood that compounds comprising a diaza-18-crown-6 macrocycle, as well as analogs, homologs, and derivatives thereof, can be employed in the present invention. Diaza-18-crown-6 macrocycle has the following general structure:

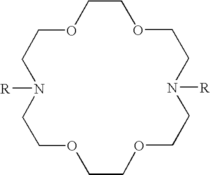

wherein R is any suitable substituent known to those of skill in the art.

It is also understood that some naturally occurring peptides, for example, alamethicin, magainin, and gramicidin can conduct cations and, therefore, exhibit antibiotic activity.

Likewise, stacked, nanotube-forming, cyclic D,L-α-peptides can exhibit significant antibiotic activity. Cyclic D,L-α-peptides, under conditions that favor hydrogen bonding between the cyclic D,L-α-peptide molecules, can self-assemble in bacterial membranes to increase membrane permeability. Suitable cyclic D,L-α-peptides are disclosed in Fernandez-Lopez, et al., “Antibacterial agents based on the cyclic D,L-α-peptide architecture,” Letters to Nature, 412, 452-329 (2001), hereby incorporated herein by reference in its entirety.

Ion channel forming compounds can optionally be used in the disclosed methods and compositions. It is understood that the present invention can be performed using other ion channel forming compounds known to those of skill in the art.

3. Oxidative Methods

Complete deactivation of spores using trace quantities of hydrogen peroxide (H₂O₂) in combination with high-pressure or supercritical CO₂, optionally in combination with other disclosed high-pressure or supercritical fluids, can be achieved. Chemically, H₂O₂ is an oxidizing agent: it produces singlet oxygen, superoxide radical, and hydroxyl radical, which are the active species that attack enzymes, membranes, DNA, etc. and eventually deactivate the microorganism. H₂O₂ contributes to the oxidation of organic molecules either in the membrane or in the interior of the cells, changing the chemical structure to render the natural biomolecules inactive, hence leading to a breakdown in cell metabolism and ultimately in cell death. Other strong oxidants, including for example, ethylene oxide and chlorine dioxide, can also be used in other sterilization and bacterial decontamination processes. Their chemical behavior is similar to hydrogen peroxide.

There can be disadvantages when using strong oxidants in sterilization. These include the toxicity, safety, and handling of these strong reagents; the possibility of corrosion or reaction with the materials of construction of the sterilization apparatus; and the non-specific oxidation reaction with sensitive biopolymers or tissues that are being sterilized. It can be, therefore, desirable to find additives that promote complete sterilization in CO₂-based fluids optionally in combination with other disclosed high-pressure or supercritical fluids, that are specific to the microorganism species and that will not adversely corrode the materials of the sterilization apparatus or degrade the instruments, tissue, or biomaterial that is being sterilized.

It is understood that the disclosed composition and methods can include oxidizing agents as a microbiocidal additive. Suitable oxidizing agents can include hydrogen peroxide, ethylene oxide, chlorine dioxide, halogens (e.g., fluorine, chlorine, bromine, and iodine), and mixtures thereof. It is also understood that other oxidizing agents known to those of skill in the art can be used in the disclosed methods and compositions.

In one aspect, the oxidizing agent can be added to the disclosed compositions and used in the disclosed methods in a concentration of greater than about 1000 ppm of the total composition. In another aspect, the concentration of oxidizing agent can be less than about 1000 ppm of the total composition, for example, less than about 800 ppm, less than about 600 ppm, less than about 500 ppm, less than about 400 ppm, less than about 300 ppm, less than about 200 ppm, less than about 100 ppm, less than about 50 ppm, less than about 25 ppm, less than about 10 ppm, or less than 5 ppm.

In a further aspect, the concentration can be from about 5 ppm to about 1000 ppm, from about 10 ppm to about 800 ppm, from about 25 ppm to about 500 ppm, from about 50 ppm to about 400 ppm, from about 50 ppm to about 300 ppm, from about 100 ppm to about 250 ppm, from about 150 ppm to about 200 ppm, about 150 ppm, about 200 ppm, or about 250 ppm.

In yet another aspect, the composition can be substantially free of oxidizing agent.

4. Other Additives

The disclosed methods and compositions can optionally include other additives. The disclosed other additives can be used, for example, in combination with both oxidative microbiocidal additives and with non-oxidative microbiocidal additives. Suitable additives include, without limitation, water, alcohols, acids, bases, or a mixture thereof.

a. Water

Water can enhance the microbiocidal effect of CO₂, optionally in combination with other disclosed high-pressure or supercritical fluids. For example, cells with low water content can show low or no deactivation. Water can swell the cells, increase the permeability of cell walls, and hydrate CO₂.

It is understood that water can be optionally included with the disclosed compositions and methods.

In one aspect, water can be added to the disclosed compositions and used in the disclosed methods in a concentration of greater than about 1000 ppm of the total composition. In another aspect, the concentration can be less than about 1000 ppm of the total composition, for example, less than about 800 ppm, less than about 600 ppm, less than about 500 ppm, less than about 400 ppm, less than about 300 ppm, less than about 200 ppm, less than about 100 ppm, less than about 50 ppm, less than about 25 ppm, less than about 10 ppm, or less than 5 ppm.

In another aspect, the concentration can be from about 100 ppm to about 1000 ppm, for example, from about 200 ppm to about 800 ppm, from about 300 ppm to about 600 ppm, or from about 400 ppm to about 500 ppm.

In yet another aspect, the composition can be substantially anhydrous.

b. Alcohols

Alcohols can act as lipid solvents and protein denaturants. Alcohols that can be optionally used in the disclosed methods and compositions include ethylene glycol, glycerol, and all C1 to C16 alcohols, including without limitation methanol, ethanol, n-propanol, isopropanol, butanol, pentanol, hexanol, heptanol, octanol, and decanol. It is also understood that mixtures of alcohols can be combined with employed in the disclosed methods and compositions.

In one aspect, alcohols can be added to the disclosed compositions and used in the disclosed methods in a concentration of greater than about 1000 ppm of the total composition. In another aspect, the concentration can be less than about 1000 ppm of the total composition, for example, less than about 800 ppm, less than about 600 ppm, less than about 500 ppm, less than about 400 ppm, less than about 300 ppm, less than about 200 ppm, less than about 100 ppm, less than about 50 ppm, less than about 25 ppm, less than about 10 ppm, or less than 5 ppm.

In another aspect, the concentration can be from about 100 ppm to about 1000 ppm, for example, from about 200 ppm to about 800 ppm, from about 300 ppm to about 600 ppm, or from about 400 ppm to about 500 ppm.

In yet another aspect, the composition can be substantially alcohol-free.

c. Variable Acidity

Change in acidity can promote deactivation of microorganisms. Lactobacillus, for example, is acid tolerant. Such bacteria use homeostasis to keep a pH difference between the cytoplasm and the medium, so they can survive in acidic conditions. However, more than 7-log reduction can be achieved by high-pressure CO₂ treatment at the same temperature and pressure over a spore suspension in distilled water at pH 6.0 (Hong, S. I. and Pyun, Y. R., “Inactivation kinetics of Lactobacillus plantarum by high pressure carbon dioxide,” J. Food Sci., 64 (1999) 728). The contrast of the ineffectiveness of an acidic environment and nitrogen with the effectiveness of CO₂ treatment of an un-acidified medium indicates that the internal pH drop is a reason for cell deactivation. Low internal pH can disturb homeostasis and proton force, and can precipitate the enzymes with acidic isoelectric points.

Even though acidity alone generally does not deactivate some microorganisms, low pH can have other effects on cells, such as weakening spore resistance to heat and attenuating resistance to destruction. Therefore, acidity can act synergistically with high-pressure CO₂, optionally in combination with other disclosed high-pressure or supercritical fluids, in deactivation.

The disclosed compositions can be made and used, and the disclosed methods performed, at specific pH values or ranges. In one aspect, the pH can be about 7. In another aspect, the pH can be about 1, about 2, about 3, about 4, about 5, about 6, about 8, about 9, about 10, about 11, about 12, about 13, or about 14.

In another aspect, the pH can be from about 2 to about 9, for example, from about 3 to about 8, from about 2 to about 5, from about 5 to about 9, or from about 4 to about 5.

It is understood that the pH can be modified by the optional addition of acids or bases that are known to those of skill in the art.

5. Techniques

a. Time

Temperature and pressure can affect the growth of microorganisms. Treatment times can vary from a few minutes (e.g., 5 min) to several days (e.g., 100 hours). Deactivation typically increases with treatment time, but the specific time required to complete deactivation depends upon the category of the microorganism (e.g., bacteria, fungus), the form of microorganism (e.g., vegetative, spore), and the treatment conditions.

Treatment time can comprise a two-stage kinetic curve. The first stage can be characterized by a slow deactivation rate, and the second stage by a fast linear deactivation. The first stage can be related to slow penetration of CO₂ into the cell walls, and is generally the controlling step of deactivation. In the second stage, CO₂, optionally in combination with other disclosed high-pressure or supercritical fluids, extracts vital components from cytoplasm or membranes.

In one aspect, the disclosed methods can be performed with a contacting step maintained, for example, for about 1 minute, about 2 minutes, about 3 minutes, about 5 minutes, about 10 minutes, about 15 minutes, about 20 minutes, about 30 minutes, about 45 minutes, about 1 hour, about 2 hours, about 3 hours, about 4 hours, about 6 hours, about 8 hours, or about 10 hours. In another aspect, the contacting time can be from about 15 minutes to about 4 hours, for example, from about 20 minutes to about 2 hours, from about 30 minutes to about 1 hour, from about 45 minutes to about 2 hours, from about 90 minutes to about 3 hours, or from about 1 hour to about 2 hours.

b. Temperature

Each microorganism has a species-specific maximum temperature. Above that temperature, proteins denature, cytoplasmic membranes collapse, and cells lyses and are deactivated. A wide range of temperatures can be employed for high-pressure fluid and CO₂ treatment, from 0° C. to 100° C. Microorganisms are generally more resistant to pressure than to temperature. A hydrostatic pressure between 1,000 to 10,000 bar can be required to deactivate bacteria (Cheftel, J. C., “Review: High-pressure, microbial inactivation and food preservation.” Food Science and Technology International 1(2-3) (1995). 75). If high-pressure CO₂ is used, the pressure requirement can be lowered below 200 bar.

Generally, deactivation can be more pronounced with increasing temperature. Higher temperature typically enhances deactivation by (a) increasing the fluidity of cell membranes, making them easier to penetrate, and (b) increasing the diffusivity of CO₂, Therefore, higher temperatures reduce the duration of the first stage of deactivation, which is thought to be diffusion-controlled. Higher temperatures can also increase the rate in the second stage. However, higher temperatures can reduce the ability of CO₂ to extract low-volatility materials and decrease CO₂ solubility in aqueous media.

In the absence of microbiocidal additives, the necessary long treatment time and high temperatures can be potential problems of the CO₂ sterilization technique. Even though a high degree of deactivation of spores can be realized, it usually requires more than 10 hours, which is not competitive with the average time of about 10 to about 15 minutes for steam sterilization. In contrast, ethylene oxide processes generally require a 15-hour cycle. Additionally, the high temperatures used (55° C.˜90° C.) can easily damage heat-sensitive materials.

When using high-pressure or supercritical CO₂, optionally in combination with other disclosed high-pressure or supercritical fluids, in one aspect, the temperature can be about 31.1° C. When using high-pressure or supercritical CO₂, in another aspect; the disclosed methods can be performed, for example, at less than about 0° C., at about 0° C., at about 5° C., at about 10° C., at about 15° C., at about 20° C., at about 25° C., at about 30° C., at about 32° C., at about 35° C., at about 40° C., at about 45° C., at about 50° C., at about 55° C., at about 60° C., at about 65° C., at about 70° C., at about 75° C., at about 80° C., at about 85° C., at about 90° C., at about 95° C., at about 100° C., or at greater than about 100° C.

In another aspect, the temperature can be from about 0° C. to about 50° C., for example, from about 0° C. to about 40° C., about 10° C. to about 50° C., about 0° C. to about 20° C., about 20° C. to about 40° C., about 30° C. to about 50° C., about 20° C. to about 30° C., or about 30° C. to about 40° C.

c. Pressure

In addition to the effects of temperature, pressure is also an important consideration in high-pressure or supercritical CO₂ sterilization. For practical applications, lower pressure improves safety, lowers capital cost, and lowers maintenance. With a decrease in pressure, the deactivation of microorganisms likewise decreases.

Kinetic behavior during deactivation can be the influenced by the efficiency of contact between CO₂, optionally in combination with other disclosed high-pressure or supercritical fluids, and the microorganism. High pressure can facilitate mass transfer through media and increase the contact between CO₂ and cells.

High pressure typically facilitates solubilization in water and penetration through cell walls, and increases density and therefore extraction power; these factors can intensify the deactivation process. There can generally be a pressure threshold below which no deactivation is observed; this pressure threshold varies with microorganism species. The D-value (the time needed to achieve 1-log reduction) of S. cerevisiae shows a steep decrease with increase in pressure from 40 to 100 bar. The duration of the earlier stage and the inactivation rate of the second stage have been found to be sensitive to pressure.

By increasing temperature and/or the pressure, less time can be required to achieve a 6-log reduction.

When using high-pressure or supercritical CO₂, optionally in combination with other disclosed high-pressure or supercritical fluids, in one aspect, the disclosed methods can be performed at pressures greater than about 73.8 bar, for example, from about 73.9 bar to about 400 bar, from about 80 bar to about 300 bar, from about 100 bar to about 200 bar, from about 200 bar to about 400 bar, from about 73.9 bar to about 200 bar, from about 100 bar to about 150 bar, from 150 bar to about 200 bar, or about 150 bar.

In another aspect, the pressure can be about 73.8 bar. In a further aspect, the pressure can be less than about 73.8 bar, for example, from about 30.4 bar to about 73.4 bar, from about 50.7 bar to about 73.4 bar, from about 60.8 bar to about 73.4 bar, or from about 70.9 bar to about 73.4 bar. In a yet further aspect, the pressure can be from about 40 bar to about 400 bar, for example, from about 40 bar to about 300 bar, from about 80 bar to about 400 bar, from about 60 bar to about 300 bar, from about 60 bar to about 200 bar, from about 60 bar to about 150 bar, or from about 40 bar to about 100 bar.

When using other high-pressure or supercritical fluids, in one aspect, the disclosed methods can be performed at about the supercritical pressure of the fluids.

When using other high-pressure or supercritical fluids, in another aspect, the pressure can be greater than the supercritical pressure of the fluid, for example, at from about 101% to about 300% of the supercritical pressure of the fluid, at from about 101% to about 200%, at from about 101% to about 150%, at from about 150% to about 300%, at from about 200% to about 300%, or at from about 150% to about 200%.

When using other high-pressure or supercritical fluids, in yet another aspect, the pressure can be less than the supercritical pressure of the fluid, for example, at from about 30% to about 99% of the supercritical pressure of the fluid, at from about 30% to about 99%, at from about 40% to about 99%, at from about 50% to about 99%, at from about 60% to about 99%, at from about 70% to about 99%, at from about 80% to about 99%, at from about 90% to about 99%, at from about 30% to about 50%, at from about 50% to about 99%, at from about 40% to about 60%, at from about 40% to about 70%, at from about 80% to about 90%, or at from about 40% to about 80%.

When using other high-pressure or supercritical fluids, in a further aspect, the pressure can be at from 30% to about 300% of the supercritical pressure of the fluids, for example, at from 30% to about 300%, at from 40% to about 300%, at from 50% to about 300%, at from 60% to about 300%, at from 70% to about 300%, at from 80% to about 300%, at from 90% to about 300%, at from 30% to about 250%, at from 30% to about 200%, at from 30% to about 150%, at from 50% to about 250%, at from 50% to about 200%, at from 50% to about 150%, at from 75% to about 300%, at from 75% to about 250%, at from 75% to about 200%, at from 75% to about 150%, or at from 75% to about 125%.

d. Depressurization Rate

A fast depressurization rate can burst cells and/or enhance mass transfer across cell membranes. Cells can be mechanically ruptured by the fast expansion of CO₂ within cells during flash discharge of pressure. Experiments at faster depressurization rates generally give higher deactivation than those at lower depressurization rates. However, other causes of cell rupture have been proposed as the cause of deactivation: a slow depressurization rate, e.g., 80 bar/h, can still achieve high deactivation.

Carbon dioxide penetration through the cell wall can be the rate controlling step. As mass transfer rate is increased, so is the deactivation effect. Fast pressurization and depressurization can enhance the transfer of CO₂ into the cells and extraction of materials from the cells.

In one aspect, the depressurization rate can be, for example, about 50 bar/h, about 100 bar/h, about 150 bar/h, about 200 bar/h, about 250 bar/h, about 300 bar/h, about 500 bar/h, about 750 bar/h, about 1000 bar/h, about 2000 bar/h, or about 3000 bar/h. In another aspect, the depressurization rate can be, for example, from about 50 bar/h to about 3000 bar/h, from about 100 bar/h to about 2000 bar/h, from about 50 bar/h to about 2000 bar/h, from about 100 bar/h to about 1000 bar/h, from about 50 bar/h to about 1000 bar/h, from about 50 bar/h to about 500 bar/h, from about 100 bar/h to about 500 bar/h, from about 200 bar/h to about 400 bar/h, from about 100 bar/h to about 400 bar/h, or from about 100 bar/h to about 300 bar/h.

e. Pressure Cycling

Pressure cycling is an alternative method to enhance deactivation while lowering the temperature and time requirements. Pressure cycling involves repeated release and compression of CO₂, optionally in combination with other disclosed high-pressure or supercritical fluids, and can enhance deactivation by enhanced cell rupture or by enhanced mass transfer. Pressure cycling can also enhance the deactivation of bacterial spores.

The enhanced deactivation of bacterial spores can also be the result of spore germination induced by high-pressure. Very high hydrostatic pressure (1000˜6000 bar) can germinate bacterial spores (e.g., B. subtilis). The mechanism can be a result of activating germinant receptors and/or cutting short germination pathways, and cycling the pressure several times can increase the efficiency of pressure-induced germination.

With pressure cycling of 30 cycles/hour, ΔP=80 bar, at 36° C. for 30 min, a 3.5-log reduction of B. subtilis spores can be achieved. Without pressure cycling, a comparable treatment at 36° C., 75 bar for 24 hours only resulted in 0.5-log reduction.

In one aspect, pressure cycling can be used in conjunction with the disclosed methods. Pressure cycling can be employed at a rate of, for example, about 0.1 cycles/hour, 0.2 cycles/hour, about 0.3 cycles/hour, about 0.5 cycles/hour, about 1 cycle/hour, about 2 cycles/hour, about 5 cycles/hour, about 10 cycles/hour, about 20 cycles/hour, about 30 cycles/hour, about 60 cycles/hour, or about 120 cycles/hour. In another aspect, the rate can be from about 0.1 cycles/hour to about 120 cycles/hour, for example, from about 0.1 cycles/hour to about 10 cycles/hour, from about 1 cycle/hour to about 60 cycles/hour, from about 20 cycles/hour to about 60 cycles/hour, from about 30 cycles/hour to about 60 cycles/hour, from about 60 cycles/hour to about 120 cycles/hour, from about 30 cycles/hour to about 90 cycles/hour, from about 90 cycles/hour to about 120 cycles/hour, from about 10 cycles/hour to about 60 cycles/hour, from about 10 cycles/hour to about 30 cycles/hour, or from about 0.1 cycles/hour to about 30 cycles/hour.

In yet another aspect, the pressure cycling change in pressure (ΔP) can be, for example, about 5 bar, about 10 bar, about 20 bar, about 30 bar, about 40 bar, about 50 bar, about 60 bar, about 70 bar, about 80 bar, about 90 bar, about 100 bar, about 150 bar, about 200 bar, about 250 bar, about 300 bar, about 400 bar, about 500 bar, or about 1000 bar. In a further aspect, the pressure cycling change in pressure (ΔP) can be, for example, from about 5 bar to about 1000 bar, from about 5 bar to about 500 bar, from about 500 bar to about 1000 bar, from 100 bar to about 200 bar, from about 200 bar to about 300 bar, from about 300 bar to about 400 bar, from about 500 bar to about 600 bar, from about 600 bar to about 700 bar, from about 700 bar to about 800 bar, from about 800 bar to about 900 bar, from about 900 bar to about 1000 bar, from about 200 bar to about 800 bar, or from about 400 bar to about 600 bar.

In a yet further aspect, pressure cycling can be omitted from the disclosed methods.

f. Agitation

The deactivation of microorganisms can be facilitated by agitation. The composition can be, for example, mixed, stirred, re-circulated, or agitated using any art recognized technique. Such agitation can be conducted prior to, during, and/or after performing the disclosed methods.

g. Energy

High-pressure and supercritical fluid mixtures can be simultaneously subjected to intense acoustic and electrostatic energy fields during deactivation to further enhance sterilization and cleaning ability. The high energy environment employed can be derived from a high-powered variable acoustic radiation source coupled with an ionizing non-uniform electric field. These high energy sources actuate specific physical and chemical changes in the dense, high-pressure or supercritical fluid chemistries, the material being processed, the unwanted residues, and the chemical agents transported in the dense fluid. This enhances cleaning, sterilization, and preservation of the materials processed.

Additional acoustic radiation can be provided by a high-powered ultrasonic generator which converts electrical energy into mechanical energy, or acoustic radiation, via a piezoelectric transducer. The transducer transmits the acoustic radiation into a dense medium such as liquids, creating intense high and low acoustic pressure waves. Intense pressure differentials can create supercritical fluid implosion cavities. Thus, the cavitation site undergoes a change from liquid state to supercritical state and back to liquid state following cavity expansion and implosion cycles (cavitation), accompanied by an overall cohesive energy change in excess of 10 MPa bar^{1/2} (32 bar^{1/2}).

In one aspect, the disclosed compositions and methods do not employ additional acoustic radiation.

Additional electrostatic energy, in the form of a non-uniform ionizing electric field, can be used to facilitate removal of contaminants from substrates through electrophoretic movement. One effect produced by the non-uniform ionizing field is charge agglomeration of submicron particles and migration of agglomerated contaminants towards a grounded internal collector plate. Another effect produced by the non-uniform electric field is electromigration of concentrated contaminants in internal pores to the more dilute dense fluid surrounding the material being processed, a process called zone electrophoresis. The electric field gradients can further enhance cleaning by causing migration of charged ionic contaminants from internal material pores towards the grounded plate.

The combination of acoustic energy and electrostatic energy can provide wide-range contaminant solubility and unidirectional contaminant mobility.

In another aspect, the disclosed compositions and methods do not employ, additional electrostatic energy. In a further aspect, the disclosed compositions and methods do not employ additional acoustic radiation and do not employ additional electrostatic energy.

6. Apparatus

The disclosed compositions and methods can be used with any apparatus suitably constructed so as to withstand the temperatures and pressures required for high-pressure and supercritical fluids and dimensioned so as to provide adequate volume to hold the material to be sterilized, the high-pressure or supercritical fluid, and any additives. To this end, any conventional sterilization apparatus known to one of skill in the art to be capable of supercritical fluid sterilization can be used with the disclosed compositions and methods. A schematic of an exemplary apparatus suitable for use with the disclosed compositions and methods is provided in FIG. 2.

Turning now to FIG. 2, an exemplary sterilization apparatus 10 is depicted. Fluid for preparation of a high-pressure or supercritical fluid is supplied to the apparatus 10 from fluid tank 20 via fluid line 15. Fluid line 15 can be an air-tight hollow tube constructed so as to withstand the required pressures for high-pressure and supercritical fluids. Fluid supply valve 25 can be positioned in fluid communication with fluid line 15 and positioned intermediate fluid tank 20 and syringe pump 30. Accordingly, fluid supply valve 25 can control the flow of fluid from fluid tank 20.

Syringe pump 30 functions to provide fluid from fluid tank 20 to apparatus 10 as well as to provide the pressures for high-pressure or supercritical fluids. In one aspect, syringe pump 30 can be calibrated to provide a selected pressure. Process valve 35 can control the flow of fluid from syringe pump 30 and is positioned along fluid line 15 between syringe pump 30 and injection valve 40. Additives can optionally be provided into the apparatus by means of injection valve 40.

Check valve 45 prevents undesired reversal of fluid flow from sterilization chamber 65 back toward pump 30 and is positioned along fluid line 15 after front valve 50. Relief valve 48 operates as an emergency vent of compressed fluid from fluid line 15 if undesired overpressure should occur and is located along fluid line 15 before front valve 50 and after injection valve 40. Front valve 50 can be closed to temporarily isolate the flow of fluid and optional additives from pump 30 and injection valve 40 from sterilization chamber 65 and is positioned along fluid line 15 between relief valve 48 and check valve 45. Overpressure disk 55 operates as a safety device to vent pressurized fluids at a pre-selected pressure.

Sterilization chamber 65 is configured so as to allow access to a hollow interior by means of portal 70, which is constructed so as to withstand the pressures of high-pressure or supercritical fluids and dimensioned to accept materials for sterilization according to the disclosed methods. In one aspect, sterilization chamber 65 can be equipped with one or more heating and/or cooling elements to regulate the temperature of the chamber 65. Preheater 60 is adapted and dimensioned to hold the pressurized fluid for sufficient residence time to attain the desired temperature maintained by the heating and/or cooling elements of sterilization chamber 65 and is located along fluid line 15 before sterilization chamber 65. Two sample valves 75 control the flow of fluids from sterilization chamber 65 leading to sample line 80, where fluids can be removed from sterilization chamber 65 without opening portal 70. Fluids can be partially or fully purged through vent 90 by means of vent valve 85.

7. Microorganisms

Previous studies on vegetative bacteria account for approximately 60% of all the studies reported, in terms of both the number of species and the number of publications. This is in accordance with the fact that food poisoning and foodborne diseases can be caused by non-sporulating vegetative bacteria. The objectives of those studies were either to preserve foods, to improve product quality, or to recover bioproducts. Different target microorganisms have been studied, depending on the practical application. To prevent food spoilage, pathogenic bacteria, such as Listeria monocytogenes, Staphylococcus aureas and Salmonella typhimurium have been used. Bacterial spores have been less frequently studied. Less than 20% of the studies are dedicated to treatment of spores, possibly because spores are highly resistant and deactivation of spores is not required for food preservation.

All of the disclosed microorganisms can be deactivated using the disclosed compositions and methods.

a. Vegetative Bacteria

Bacteria are generally categorized into two major groups, gram-positive and gram-negative bacteria, according to their responses to the gram stain. The different response to the gram stain derives from differing peptidoglycan content. Gram-positive cell walls are simple in structure, but have thick peptidoglycan layers (10 to 20 layers thick, as much as 90% of the cell wall), which make the cell walls strong and robust. However, gram-negative cells have complex cell wall structures but much thinner peptidoglycan layers (only 1 to 2 layers thick, about 10% of the cell wall). Therefore, the gram-positive cells are generally stronger, less likely to be broken mechanically, and are less permeable than the gram-negative cells.

Generally, gram-positive bacteria have been more difficult to deactivate than gram-negative bacteria. However, gram-negative bacteria are not always more susceptible to high-pressure CO₂ treatment than gram-positive bacteria. Generally, gram-positive species show resistance greater than, or at least equal to, gram-negative species. Though there is a difference in sensitivity between the gram-positive and the gram-negative vegetative bacteria, both are susceptible to high-pressure or supercritical CO₂ treatment, optionally in combination, with other disclosed high-pressure or supercritical fluids.

b. Endospores

A spore (endospore) is the highly resistant dormant form of various bacilli and clostridia. Spores are formed within vegetative cells under harsh environments such as poor nutrition; through a sporulation process. They are highly resistant to heat, UV radiation, free radicals, and chemicals because of their unique structures. Compared to a vegetative cell which contains on the order of 80˜90% water, the spore core is highly dehydrated (only 10˜25% water content), making it very resistant to heat and chemicals. The Ca²⁺ dipicolinic acid complex and small acid-soluble protein (SASP), which bind to DNA, increase spore resistance to heat, desiccation, and UV radiation. The outside of the spore core, a thick, loosely cross-linked peptidoglycan layer, is referred to as the spore cortex, which prevents hydration of the spore core. The outmost structure is the multilayered spore coat, which can be a permeability barrier to chemicals such as chloroform and lysozyme.

Because spores are highly resistant to heat, chemicals and radiation, extreme temperatures (121° C. steam), UV radiation, or highly oxidative chemicals, e.g., ethylene oxide, are used for sterilization. Spore survivability is a standard assay to test sterilization equipment. The most frequently used model organisms are G. stearothermophilus, which is used to test steam and hydrogen peroxide sterilizers, B. subtilis, which can be used to test dry heat and ethylene oxide sterilizers and B. pumilus, which can be used to test radiation sterilizers. B. pumilus ATCC 27142 spores are an industrial standard for testing radiation sterilization.

Spores can be highly resistant to dense-phase CO₂ treatment. Vegetative G. stearothermophilus cells have been reduced by more than 6-log after 1.5-hour exposure to CO₂ at 28 bar and 25° C. However, even with two-hour exposure to pure CO₂ at 200 bar and 35° C., 80% of G. stearothermophilus spores remained viable. Even with the addition of ethanol or acetic acid, less than 60% of G. stearothermophilus spores were deactivated.

8. Quantifying Deactivation

Several techniques can be used to quantify the number of surviving cells and to characterize changes in integrity of the cell membrane, the permeability of the cell wall, and morphological changes. One technique is a standard agar-plate counting method. The treated cell suspension can be serially diluted and inoculated to agar plates. The number of colonies on each Petri dish is counted after incubation for a certain time, and then the colony count is converted to the cell concentration in the original cell suspension. Plate counting can be effective way to quantify the number of viable cells, but generally does not reveal mechanistic information.

To visually observe structure changes in cells, Scanning Electron Microscopy (SEM) and Transmission Electron Microscopy (TEM) can be used to observe surface and internal structure changes induced by high-pressure CO₂ treatment. Burst cells, wrinkles and holes on the cell surface, as well as intact cell surfaces can be observed.

UV absorbance and protein analysis can also be used to determine loss of cell content as a result of CO₂ treatment of wet cell slurries. If the cell membranes are damaged, internal components such as lipids, amino acids, and peptides can be detectable in the media. The presence of these materials can be detected by UV absorbance and protein analysis. UV studies reveal an increase in absorbance after CO₂ treatment.

Proteins can be analyzed using a bicinchoninic acid assay kit, and other techniques can also be used, such as Coulter counting to measure number and size of microorganism cells, enzyme activity assay to determine CO₂ effects on enzymes, and dye uptake to study membrane damage.

The degree of deactivation can be calculated from the results of measuring the number of spores on an untreated material and the number of spores on a treated material with the following equation:

$$Log Reduction = log ( \frac{number of untreated microorganisms}{number of surviving microorganisms after treatment} )$$

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a. Materials

B. pumilus ATCC 27142 spore strips (mean recovery: 3.5×10⁶ cfu/strip) and B. atrophaeus ATCC 9372 spore strips (mean recovery: 1.8×10⁶ cfu/strip) were purchased from Raven Biological Laboratories, Inc., Omaha, Nebr. B. anthracis ΔSterne-1 spore strips were prepared in-house (mean recovery: 2.8×10⁶ cfu/strip). Difco™ tryptic soy agar (Sparks, Md.) and 30% hydrogen peroxide aqueous solution (Fair Lawn, N.J.) were obtained from Fisher Scientific. Anhydrous CO₂ (purity>99.8%) was obtained National Specialty Gases (Durham, N.C.) was used.

b. Equipment

High-pressure treatment experiments were conducted in an ISCO SFX 2-10 two cartridge fluid extractor extraction system (Lincoln, Nebr.) depicted in FIG. 2. This system can include two ISCO D260 high-pressure syringe pumps, which can be controlled by an ISCO series D pump controller. The pumps supply CO₂ to the system from a CO₂ tank through the process valve, a Valco Instruments (Houston, Tex.) six-port liquid injection valve, then into the pressure cartridge. At the end of an experiment, CO₂ within the pressure cartridge was released rapidly through the 1/16 inch vent valve.

In this example, liquid additives, such as ethanol, isopropanol, and hydrogen peroxide, were introduced into the system in one of two ways. They were either pipetted directly onto spore strips, or were delivered with the CO₂ using the Valco Instruments six-port valve. The valve was used to quantitatively and instantaneously inject a small amount of liquid additives directly into the CO₂ stream. The valve was switched between the sample-loading position and the injection position by turning the rotor tab. The loop was detachable, which allows different volume loops to be installed with different injection volumes. A 5 μl loop was used in this example.

To prevent cross-contamination, pressure cartridges were steam-autoclaved before each experiment. The transfer of spore strips in and out of the pressure cartridge and quantification of survived spores were performed under aseptic conditions.

c. Sterilization Procedure

The two-cartridge ISCO apparatus allowed use for either one or two pressure cartridges for a given experiment. The choice of using one or both cartridges was determined by the experimental conditions. For example, if injection of a liquid additive through the six-port valve was required, only one cartridge was used for pressure treatment to ensure that the additive was injected quantitatively into this cartridge. The other cartridge was either left empty and not pressurized, or was used for a depressurized control experiment.

The following procedure describes an experiment using only one pressure cartridge, with a liquid additive injection.

Prior to the pressure treatment, four spore strips were aseptically transferred from their glassine envelopes into one steam-autoclaved, dry ten-milliliter pressure cartridge under a Bunsen burner. When the temperature of the extractor attained the selected temperature (40° C. in this example, but other temperatures can be used), the pressure cartridge was inserted into the extractor, then the cartridge was flushed with CO₂ (˜55 bar) for approximately five seconds. After venting this CO₂ to atmospheric pressure, the six-port valve was switched from the loading position to the injecting position. The extractor vent valve was closed, and the system was filled with CO₂ to the experimental pressure using the ISCO pumps. The additive in the sample loop was thus transported quantitatively into the cartridge by the flowing CO₂. The system pressure in this example was monitored and recorded with a Digiquartz portable precision pressure transducer (Paroscientific, Inc., model 740, Redmond, Wash.) with a computer interface.

The pressure was maintained for the predetermined amount of time, followed by quickly depressurizing the system to atmospheric pressure by opening the vent valves. After the predetermined process time, CO₂ was vented until the cartridge reached atmospheric pressure. The cartridge containing the spore strips was immediately removed from the extractor.

d. Colony Counting

The treated spore strips were aseptically transferred under a Bunsen burner into a sterile VWR Filtra Stomacher bag (LABPLAS Inc., Ste-Julie, Canada). One hundred milliliters of steam-autoclaved, room-temperature de-ionized water was poured into the bag. The Filtra bag was then placed in a Stomacher 400 blender (Seward Limited, Norfolk, UK) to pulverize the spore strips at a speed of 260 rpm. The Filtra bag was checked every ten minutes until the spore strips were disintegrated into a homogeneous pulp after about twenty minutes. From the pulp, two 30 mL aliquots were pipetted into two 40 mL vials. The two 40 mL vials were then heat-shocked in a NESLAB RTE7 circulator (Thermo Electron Corporation, Waltham, Mass.) (80-85° C.) for ten minutes.

Immediately after heat-shock, the vials were quenched in an ice water bath (0-5° C.) for ten minutes. Then the contents from the two 40 mL vials were used to quantify the surviving cells employing a serial dilution-agar pour plate procedure. Each spore suspension was diluted by a factor of ten until a dilution of 10-5 was attained. A spore suspension of each dilution was plated on three Petri dishes.

The number of colony forming units (cfu) on each Petri dish was counted after 24 and 48 hours incubation at 30-35° C. The average number of cfu was calculated from the three Petri dishes at each dilution and converted to the corresponding number of surviving spores on one spore strip using the corresponding dilution factor. Finally, the log reduction of B. pumilus spores on one spore strip was calculated using the following equation:

$$Log Reduction = log ( \frac{number of untreated spores}{number of surviving spores after treatment} )$$

e. Results

The results obtained from employing five microliters of each of the following additives, hydrogen peroxide, ethanol, isopropanol, and bulk water, at different conditions are compared in Table 2. Neither bulk water, 5 μL of 70% ethanol, nor 5 μL of 70% isopropanol resulted in the required log reduction. However, hydrogen peroxide proved highly effective in deactivating dry B. pumilus spores. Additional hydrogen peroxide (5 μL) injected experiments were conducted at 40° C., 50° C. and 60° C., at 276 bar for 4 hours. Even when B. pumilus spores were treated at a temperature as low as 40° C., the hydrogen peroxide additive resulted in a 4.47-log reduction.

Comparatively, this result was statistically much higher than the reduction obtained from water-wetted spore strips at a much higher temperature (3.06-log reduction at 60° C., 3.02-log reduction at 80° C., p<0.05). At 60° C., the hydrogen peroxide injection method gave complete deactivation in two of the three replicate experiments, and the third replicate showed one colony in one of the three undiluted plates. Five micro-liters of H₂O₂ correspond to a concentration of approximately 200 ppm H₂O₂ in the 10 mL pressure cartridge.

The concentration of hydrogen peroxide in applications such as contact lens cleaning is usually 3%, but research shows that lenses treated with only 200 ppm aqueous H₂O₂ can cause discomfort. Hence, a lower concentration of hydrogen peroxide can be preferred in practice. In comparing the results of five micro-liters of H₂O₂ injected experiments at different temperatures, the largest increase in the log reduction was observed on going from 50° C. to 60° C., 4.60 to 6.28, respectively. However, even though a high degree of deactivation can be achieved by increasing temperature, this would not be desirable for treatment of heat-sensitive materials and devices. For hydrogen peroxide, decreasing the treatment time at 60° C. from 4 hours to 2 hours lowered the log reduction from 6.28 for 4 hours to 4.45 for 2 hours.

The results depicted in Table 2 with H₂O₂ demonstrate that a small quantity of an additive (5 μl of hydrogen peroxide vs. ˜1 ml of water) can be used to achieve a high degree of deactivation. Using H₂O₂ can be both economical and safe and no post-processing would be required to remove the water and oxygen generated from H₂O₂ decomposition. It also makes CO₂ sterilization possible under dry conditions.

TABLE 2
LOG REDUCTION ACHIEVED WITH DIFFERENT ADDITIVES

| | TEMPERA- | PRES- | | |
|---|---|---|---|---|
| | TURE | SURE | DURATION | LOG |
| ADDITIVE | [° C.] | [BAR] | [HR] | REDUCTION |
| BULK WATER | 50 | 276 | 4 | 0.58 ± 0.04 |
| BULK WATER | 60 | 276 | 4 | 3.06 ± 0.17 |
| BULK WATER | 80 | 276 | 4 | 3.02 ± 0.07 |
| 5 μL 70% | 40 | 276 | 4 | 0.27 ± 0.16 |
| ETHANOL | | | | |
| 5 μL 70% | 40 | 276 | 4 | 0.17 ± 0.06 |
| ISOPROPANOL | | | | |
| 5 μL 30% H₂O₂ | 40 | 276 | 4 | 4.47 ± 0.64 |
| 5 μL 30% H₂O₂ | 50 | 276 | 4 | 4.60 ± 0.02 |
| 5 μL 30% H₂O₂ | 60 | 276 | 4 | 6.28 ± 0.46 |
| 5 μL 30% H₂O₂ | 60 | 276 | 2 | 4.45 ± 0.31 |

However, it is generally less desirable to soak medical devices in liquid before sterilization. The present results show that as little as 200 ppm H₂O₂ in CO₂ can boost the log reduction to 4.47 at 40° C. and 6.28 at 60° C. respectively.

B. pumilus spore strips were treated with dense-phase CO₂ using different additives (water, ethanol, isopropanol, and hydrogen peroxide), temperatures (40˜80° C.), pressures (Pₐₜₘ, 103 bar, 276 bar), and time increments (1˜6 hours). At 40° C., neither bulk water, nor ethanol, nor isopropanol were effective additives. Water can be made more effective by using higher temperatures (up to 80° C.) and increasing treatment time (up to six hours). However, it is generally less desirable to use high temperatures, which can damage heat-sensitive materials and devices. A long treatment time can increase overall expense.

B. atrophaeus and B. anthracis spore strips were also treated with supercritical CO₂ using five microliters of 30% H₂O₂ at 40° C., 276 bar, and 4 hours. Log reductions of 6.25 and 5.74 were measured with B. atrophaeus and B. anthracis spores, respectively.

In this example, five microliters of 30% H₂O₂ was the most effective additive among the additives examined. This small amount of H₂O₂ is equivalent to 200 ppm in a 10 mL pressure cartridge, allowing for dry sterilization of medical devices. Moreover, use of H₂O₂ as an additive indicates a new possible mechanism of deactivation, CO₂ facilitated. Treatment at a lower pressure yielded a lower log reduction, which can be a consequence of lower CO₂ density. The effect of exposure time on the deactivation with bulk water in conjunction with high-pressure CO₂ was nonlinear, with three different stages of deactivation.

### 2. Example 2 (Prophetic)

The present invention can also be performed with high-pressure carbon dioxide and at least one non-oxidative additive. Materials and equipment can be the same as those used in Example 1. Non-oxidative additive, for example L-alanine, can be introduced into the system by either direct addition onto spore strips or by delivery with the CO₂.

a. Sterilization Procedure

At least one spore strip can be aseptically transferred into a steam-autoclaved, dry ten-milliliter pressure cartridge under a Bunsen burner. The pressure cartridge can be then inserted into the extractor, and the cartridge can be then flushed with CO₂, optionally in combination with other disclosed high-pressure or supercritical fluids. After venting this CO₂ to atmospheric pressure, the six-port valve can be switched from the loading position to the injecting position. With the extractor vent valve closed, and the system can be filled with CO₂ to the experimental pressure. The additive in the sample loop can be thus transported quantitatively into the cartridge by the flowing CO₂.

The pressure can be maintained for the predetermined amount of time, followed by quickly depressurizing the system to atmospheric pressure by opening the vent valves. After the predetermined process time, CO₂ can be vented until the cartridge reaches atmospheric pressure. The cartridge containing the spore strips can then be removed from the extractor.

b. Colony Counting

The treated spore strip(s) can then be aseptically transferred, pulverized, and disintegrated, as in Example 1. After heat-shock and ice bath quench, the sample(s) can be used to quantify the surviving cells employing a serial dilution-agar pour plate procedure.

The number of colony forming units (cfu) on each Petri dish can be counted after 24 and 48 hours incubation at 30-35° C., and the log reduction of spores on the spore strip(s) can be calculated using the following equation:

$$Log Reduction = log ( \frac{number of untreated spores}{number of surviving spores after treatment} )$$

c. Results

The log reduction will typically be at least 2-log for methods employing high-pressure carbon dioxide and non-oxidative additives.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for sterilizing a material having microorganisms to be inactivated comprising the steps of:
a. contacting the material with a mixture comprising a non-oxidative microbiocidal additive and high-pressure carbon dioxide or supercritical carbon dioxide, and
b. maintaining the contact for a period of time effective to achieve a degree of inactivation of the microorganisms exceeding 2 log orders.

2. The method of claim 1, wherein the additive comprises a spore germinant.

3. The method of claim 2, wherein the spore germinant comprises L-alanine, dipicolinic acid, asparagine, glucose, fructose, a potassium ion, or a mixture thereof.

4. The method of claim 1, wherein the additive comprises a peptidoglycan hydrolyzer.

5. The method of claim 4, wherein the peptidoglycan hydrolyzer comprises lysozyme, autolysin, lysostaphin, HEL96-116 peptide, or a mixture thereof.

6. The method of claim 1, wherein the additive comprises an ion channel forming compound.

7. The method of claim 6, wherein the ion channel forming compound comprises a diaza-18-crown-6 macrocycle.

8. The method of claim 6, wherein the ion channel forming compound comprises C12 macrocyclic hydraphile.

9. The method of claim 6, wherein the ion channel forming compound comprises alamethicin, magainin, gramicidin, or a mixture thereof.

10. The method of claim 6, wherein the ion channel forming compound comprises a cyclic D,L-α-peptide.

11. The method of claim 1, wherein the contact is maintained for a period of time effective to achieve a degree of inactivation of the microorganisms exceeding 3 log orders.

12. The method of claim 1, wherein the contact is maintained for a period of time effective to achieve a degree of inactivation of the microorganisms exceeding 4 log orders.

13. The method of claim 1, wherein the contact is maintained for a period of time effective to achieve a degree of inactivation of the microorganisms exceeding 6 log orders.

14. The method of claim 1, wherein the mixture further comprises an oxidative microbiocidal additive.

15. The method of claim 14, wherein the oxidative microbiocidal additive comprises hydrogen peroxide, ethylene oxide, ozone, chlorine dioxide, a halogen, or a mixture thereof.

16. The method of claim 1, wherein the mixture further comprises water.

17. The method of claim 1, wherein the mixture further comprises an alcohol.

18. The method of claim 1, wherein the mixture further comprises a second high-pressure or supercritical fluid.

19. The method of claim 18, wherein the second high-pressure or supercritical fluid comprises nitrogen, nitrous oxide, ethylene, argon, tetrafluoroethane, or a mixture thereof.

20. The method of claim 1, wherein the additive comprises L-alanine, dipicolinic acid, asparagine, glucose, fructose, a potassium ion, or a mixture thereof and wherein the contact is maintained for a period of time effective to achieve a degree of inactivation of the microorganisms exceeding 6 log orders.

21. The method of claim 1, wherein the additive comprises lysozyme, autolysin, lysostaphin, HEL96-116 peptide, or a mixture thereof and wherein the contact is maintained for a period of time effective to achieve a degree of inactivation of the microorganisms exceeding 6 log orders.

22. The method of claim 1, wherein the additive comprises diaza-18-crown-6 macrocycle, C12 macrocyclic hydraphile, alamethicin, magainin, gramicidin, or a mixture thereof and wherein the contact is maintained for a period of time effective to achieve a degree of inactivation of the microorganisms exceeding 6 log orders.

23. A method for sterilizing a material having microorganisms to be inactivated comprising the steps of:
a. contacting the material with a mixture comprising a non-oxidative microbiocidal additive and a high-pressure or supercritical fluid, and
b. maintaining the contact for a period of time effective to achieve a degree of inactivation of the microorganisms exceeding 2 log orders.

24. The method of claim 23, wherein the fluid comprises carbon dioxide, nitrogen, nitrous oxide, ethylene, argon, tetrafluoroethane, or a mixture thereof.

25. The method of claim 23, wherein the additive comprises a spore germinant.

26. The method of claim 25, wherein the spore germinant comprises L-alanine, dipicolinic acid, asparagine, glucose, fructose, a potassium ion, or a mixture thereof.

27. The method of claim 23, wherein the additive comprises a peptidoglycan hydrolyzer.

28. The method of claim 27, wherein the peptidoglycan hydrolyzer comprises lysozyme, autolysin, lysostaphin, HEL96-116 peptide, or a mixture thereof.

29. The method of claim 23, wherein the additive comprises an ion channel forming compound.

30. The method of claim 29, wherein the ion channel forming compound comprises a diaza-18-crown-6 macrocycle.

31. The method of claim 29, wherein the ion channel forming compound comprises C12 macrocyclic hydraphile.

32. The method of claim 29, wherein the ion channel forming compound comprises alamethicin, magainin, gramicidin, or a mixture thereof.

33. The method of claim 29, wherein the ion channel forming compound comprises a cyclic D,L-α-peptide.

34. The method of claim 23, wherein the contact is maintained for a period of time effective to achieve a degree of inactivation of the microorganisms exceeding 3 log orders.

35. The method of claim 23, wherein the contact is maintained for a period of time effective to achieve a degree of inactivation of the microorganisms exceeding 4 log orders.

36. The method of claim 23, wherein the contact is maintained for a period of time effective to achieve a degree of inactivation of the microorganisms exceeding 6 log orders.

37. The method of claim 23, wherein the mixture further comprises an oxidative microbiocidal additive.

38. The method of claim 37, wherein the oxidative additive comprises hydrogen peroxide, ethylene oxide, chlorine dioxide, a halogen, or a mixture thereof.

39. The method of claim 23, wherein the mixture further comprises water.

40. The method of claim 23, wherein the mixture further comprises an alcohol.

41. A method for sterilizing a material having microorganisms to be inactivated comprising the steps of:
a. contacting the material with a mixture comprising an oxidative microbiocidal additive and supercritical carbon dioxide, and
b. maintaining the contact for a period of time effective to achieve a degree of inactivation of the microorganisms exceeding 2 log orders.

42. The method of claim 23, wherein the oxidative microbiocidal additive comprises hydrogen peroxide, ethylene oxide, chlorine dioxide, a halogen, or a mixture thereof.

43. The method of claim 23, wherein the oxidative microbiocidal additive is present in a concentration of less than 1000 ppm of the total composition.

44. The method of claim 23, wherein the oxidative microbiocidal additive is present in a concentration of less than 200 ppm of the total composition.

45. The method of claim 23, wherein the contact is maintained for a period of time effective to achieve a degree of inactivation of the microorganisms exceeding 3 log orders.

46. The method of claim 23, wherein the contact is maintained for a period of time effective to achieve a degree of inactivation of the microorganisms exceeding 4 log orders.

47. The method of claim 23, wherein the contact is maintained for a period of time effective to achieve a degree of inactivation of the microorganisms exceeding 6 log orders.

48. The method of claim 23, wherein the mixture further comprises water.

49. The method of claim 23, wherein the mixture further comprises an alcohol.

50. The method of claim 23, wherein the mixture further comprises a second high-pressure or supercritical fluid.

51. The method of claim 50, wherein the second high-pressure or supercritical fluid comprises nitrogen, nitrous oxide, ethylene, argon, tetrafluoroethane, or mixtures thereof.

52. The method of claim 23, wherein the mixture is not subjected to increased acoustic and electrostatic energy radiation during the contacting step.

53. The method of claim 23, wherein the oxidative microbiocidal additive is hydrogen peroxide and is present in a concentration of less than 200 ppm of the total composition and wherein the contact is maintained for a period of time effective to achieve a degree of inactivation of the microorganisms exceeding 6 log orders.

54. A composition for sterilizing a material, comprising:
a. a non-oxidative microbiocidal additive and
b. high-pressure carbon dioxide or supercritical carbon dioxide.

55. The composition of claim 54, wherein the additive comprises a spore germinant.

56. The composition of claim 55, wherein the spore germinant comprises L-alanine, dipicolinic acid, asparagine, glucose, fructose, a potassium ion, or a mixture thereof.

57. The composition of claim 54, wherein the additive comprises a peptidoglycan hydrolyzer.

58. The composition of claim 57, wherein the peptidoglycan hydrolyzer comprises lysozyme, lysostaphin, autolysin, HEL96-116 peptide, or a mixture thereof.

59. The composition of claim 54, wherein the additive comprises an ion channel forming compound.

60. The composition of claim 59, wherein the ion channel forming compound comprises a diaza-18-crown-6 macrocycle.

61. The composition of claim 59, wherein the ion channel forming compound comprises C12 macrocyclic hydraphile.

62. The composition of claim 59, wherein the ion channel forming compound comprises alamethicin, magainin, gramicidin, or a mixture thereof.

63. The method of claim 59, wherein the ion channel forming compound comprises a cyclic D,L-α-peptide.

64. The composition of claim 54, wherein the additive is present in a concentration of less than 1000 ppm of the total composition.

65. The composition of claim 54, wherein the additive is present in a concentration of less than 200 ppm of the total composition.

66. The composition of claim 54, further comprising an oxidative microbiocidal additive.

67. The composition of claim 66, wherein the oxidative microbiocidal additive comprises hydrogen peroxide, ethylene oxide, chlorine dioxide, a halogen, or a mixture thereof.

68. The composition of claim 54, further comprising water.

69. The composition of claim 54, further comprising an alcohol.

70. The composition of claim 54, further comprising a second high-pressure or supercritical fluid.

71. The composition of claim 70, wherein the second high-pressure or supercritical fluid comprises nitrogen, nitrous oxide, ethylene, argon, tetrafluoroethane, or mixtures thereof.

72. A composition for sterilizing a material, comprising:
a. a non-oxidative microbiocidal additive and
b. a high-pressure or supercritical fluid.

73. The composition of claim 72, wherein the fluid comprises carbon dioxide, nitrogen, nitrous oxide, ethylene, argon, tetrafluoroethane, or a mixture thereof.

74. The composition of claim 72, wherein the additive comprises a spore germinant.

75. The composition of claim 74, wherein the spore germinant comprises L-alanine, dipicolinic acid, asparagine, glucose, fructose, a potassium ion, or a mixture thereof.

76. The composition of claim 72, wherein the additive comprises a peptidoglycan hydrolyzer.

77. The composition of claim 76, wherein the peptidoglycan hydrolyzer comprises lysozyme, lysostaphin, HEL96-116 peptide, or a mixture thereof.

78. The composition of claim 72, wherein the additive comprises an ion channel forming compound.

79. The composition of claim 78, wherein the ion channel forming compound comprises a diaza-18-crown-6 macrocycle.

80. The composition of claim 78, wherein the ion channel forming compound comprises C12 macrocyclic hydraphile.

81. The composition of claim 78, wherein the ion channel forming compound comprises alamethicin, magainin, gramicidin, or a mixture thereof.

82. The method of claim 78, wherein the ion channel forming compound comprises a cyclic D,L-α-peptide.

83. The composition of claim 72, wherein the contact is maintained for a period of time effective to achieve a degree of inactivation of the microorganisms exceeding 3 log orders.

84. The composition of claim 72, wherein the contact is maintained for a period of time effective to achieve a degree of inactivation of the microorganisms exceeding 4 log orders.

85. The composition of claim 72, wherein the contact is maintained for a period of time effective to achieve a degree of inactivation of the microorganisms exceeding 6 log orders.

86. The composition of claim 72, wherein the mixture further comprises an oxidative microbiocidal additive.

87. The composition of claim 86, wherein the oxidative additive comprises hydrogen peroxide, ethylene oxide, ozone, chlorine dioxide, a halogen, or a mixture thereof.
